(19) United States
(12) Patent Application Publication
(10) Pub. No.: US 2005/0159414 A1
(43) Pub. Date: Jul. 21, 2005

(54) Use of substituted pyrimido[5,4-D]pyrimidines for the treatment of respiratory diseases

(75) Inventors: Peter Nickolaus, Warthausen (DE); Christopher J. Montague Meade, Maselheim (DE); Domnic Martyres, Biberach (DE); Juergen Mack, Biberach (DE); Birgit Jung, Laupheim (DE); Horst Dollinger, Schemmerhofen (DE); Georg Dahmann, Attenweiler (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/034,108

(22) Filed: Jan. 12, 2005

Related U.S. Application Data
(60) Provisional application No. 60/541,394, filed on Feb. 3, 2004.

(30) Foreign Application Priority Data
Jan. 17, 2004 (DE) 10 2004 002 557.6

(51) Int. Cl. A61K 31/541

(52) U.S. Cl. 514/227.8

(57) ABSTRACT

The invention relates to the use of pyrimido[5,4-d]pyrimidines for the treatment of inflammatory and obstructive respiratory complaints, preferably asthma or COPD, as well as pharmaceutical compositions which contain these compounds.

This application claims priority benefit of U.S. Application Ser. No. 60/541,379, filed Feb. 3, 2004, which claims benefit of German Application DE 10 2004 002 557.6 filed Jan. 17, 2004 each of which is hereby incorporated by reference in its entirety.

The invention relates to the use of pyrimido[5,4-d]pyrimidines for the treatment of inflammatory and obstructive respiratory complaints, preferably asthma or COPD, as well as pharmaceutical compositions which contain these compounds.

### BACKGROUND TO THE INVENTION

Inflammatory and obstructive respiratory complaints belong to the group of progressive respiratory complaints which are characterised by breathing problems, among other things. These breathing problems are usually associated with chronic inflammation of the airways involving different cells, particularly macrophages, neutrophils and CD8 T lymphocytes.

The aim of the present invention is to provide a medicament for the treatment of inflammatory and obstructive respiratory complaints. A further aim of the present invention is to provide medicaments for the treatment of inflammatory and obstructive respiratory complaints which are characterised by fewer side effects, particularly emesis and nausea.

Pyrimido[5,4-d]pyrimidines are known as active substances with an antiproliferative activity. DE 1151806 describes pyrimido[5,4-d]pyrimidines as coronary dilatators. EP 23559 describes 2-(perhydro-1,4-diazino)pyrimido[5,4-d]pyrimidines as having an inhibitory effect on the aggregation of cancer cells carried into the bloodstream. EP 55444 describes trisubstituted pyrimido[5,4-d]pyrimidines as compounds which in addition to having a hypotensive and cardiotonic activity also have an inhibitory effect on the aggregation of cancer cells carried into the bloodstream.

### DESCRIPTION OF THE INVENTION

One aspect of the present invention is directed to the use of pyrimido[5,4-d]pyrimidines for treating respiratory complaints, particularly inflammatory and obstructive respiratory complaints.

Another aspect of the present invention is directed to the use of pyrimido[5,4-d]pyrimidines to prepare a medicament for the treatment of respiratory complaints wherein only minor side-effects occur.

It is preferable to use substituted pyrimido[5,4-d]pyrimidines to prepare a medicament for the treatment of inflammatory or obstructive respiratory complaints, particularly preferably is COPD or asthma.

It is particularly preferable to use substituted pyrimido[5,4-d]pyrimidines to prepare a medicament for the treatment of inflammatory or obstructive respiratory complaints, particularly preferably COPD or asthma while at the same time reducing the side effects, particularly emesis or nausea.

It is preferable to use compounds of general formula 1 to prepare a medicament for the treatment of the above-mentioned respiratory complaints 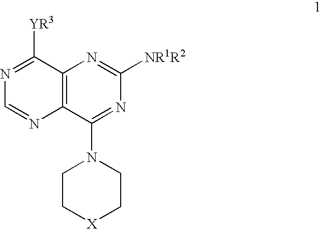
wherein
R¹ and R² denotes H or a group selected from among —C₁₋₆-alkyl, —C₃₋₈-cycloalkyl and —C₁₋₆-alkyl-O—C₁₋₆-alkyl, which may optionally be mono- or polysubstituted by one or more groups selected from among aryl, —CF₃, —CN, —CONR⁴R⁵, —COOR⁴, —COR⁶, halogen, heteroaryl, heterocycle, —NO₂, —NR⁴COR⁶, —NR⁴R⁵, —NR⁴SO₂R⁴, —OR⁴, —SO₂NR⁴R⁵, —SO₂R⁴, —SOR⁴ and —SR⁴ or
R¹ and R² together with the nitrogen form a ring which may optionally be mono- or polysubstituted by one or more groups selected from among aryl, —C₁₋₆-alkyl-OH, —CF₃, —CN, —CONR⁴R⁵, —COOR⁴, —COR⁶, halogen, heteroaryl, heterocycle, —NO₂, —NR⁴COR⁶, —NR⁴R⁵, —NR⁴SO₂R⁴, —O—C₁₋₆-alkyl, —OR⁴, —SO₂NR⁴R⁵, —SO₂R⁴, —SOR⁴ and —SR⁴;
R⁴ and R⁵ independently of one another denote H, —C₁₋₆-alkyl;
R⁶ denotes H, —C₁₋₆-alkyl, —C₁₋₆-alkyl-CO—C₁₋₆-alkyl, heteroaryl, heterocycle, —NR⁴R⁵;
R³ denotes H or a group selected from among —C₁₋₈-alkyl, —C₃₋₈-cycloalkyl, —C₁₋₆-alkyl-R⁷ and phenyl which may optionally be anellated or may be mono- or polysubstituted by one or more groups selected from among —CF₃, —CN, —CONR⁴R⁵, —COOR⁴, —COR⁴, halogen, heteroaryl, heterocycle, —NO₂, —NR⁴COR⁴, —NR⁴R⁵, —NR⁴SO₂R⁴, —OR⁴, —SO₂NR⁴R⁵, —SO₂R⁴, —SOR⁴ and —SR⁴;
R⁷ denotes heteroaryl, heteroaryl anellated with heterocycle, heterocycle or phenyl, which may optionally be mono- or polysubstituted by one or more substituents selected from among —C₁₋₆-alkyl, —O—C₁₋₆-alkyl, halogen, —NO₂ and —CF₃;
X denotes —S—, —S(O)—, —S(O₂)— and
Y denotes —NR⁴—, —S—, —O—;
as well as pharmacologically acceptable acid addition salts, tautomeric and isomeric forms or mixtures and individual geometric or optical isomers, particularly racemic or non-racemic mixtures of the isomers thereof.

It is particularly preferable to use compounds of general formula 1 to prepare a medicament for the treatment of the above-mentioned respiratory complaints, wherein
R¹ and R² denote H or a group selected from among —C₁₋₆-alkyl, —C₃₋₈-cycloalkyl and —C₁₋₆-alkyl-O—C₁₋₆-alkyl, which may optionally be mono- or polysubstituted by one or more groups selected from among —CONR⁴R⁵, —COOR⁴, —COR⁶, halogen, heteroaryl, heterocycle, —NR⁴COR⁶, —NR⁴R⁵ and —OR⁴ or
R¹ and R² together with the nitrogen form a ring which may optionally be mono- or polysubstituted by one or more groups selected from among —C₁₋₆-alkyl-OH, —CONR⁴R⁵, —COOR⁴, —COR⁶, —NR⁴COR⁶, —NR⁴R⁵, —NR⁴SO₂R⁴, —O—C₁₋₆-alkyl, —OR⁴, —SO₂NR⁴R⁵, —SO₂R⁴, —SOR⁴ and —SR⁴;
R⁴ and R⁵ independently of one another denote H, —C₁₋₆alkyl;
R⁶ denotes H, —C₁₋₆-alkyl, —C₁₋₆-alkyl-CO—C₁₋₆-alkyl, heteroaryl, heterocycle, —NR⁴R⁵;
R³ denotes H or a group selected from among —C₁₋₈-alkyl, —C₃₋₈-cycloalkyl, —C₁₋₆-alkyl-R⁷ and phenyl which may optionally be anellated or may be mono- or polysubstituted by one or more groups selected from among —CF₃, —CN, —CONR⁴R⁵, —COOR⁴, —COR⁴, halogen, —NO₂, —NR⁴COR⁴, —NR⁴R⁵, —NR⁴SO₂R⁴, —OR⁴ and —SR⁴;
R⁷ denotes heteroaryl, heteroaryl anellated with heterocycle, heterocycle or phenyl, which may optionally be mono- or polysubstituted by one or more substituents selected from among —C₁₋₆-alkyl, —O—C₁₋₆-alkyl, halogen, —NO₂ and —CF₃;
X denotes —S—, —S(O)—, —S(O₂)—;
Y denotes —NR⁴—, —S—, —O—;
and the pharmacologically acceptable acid addition salts, tautomeric and isomeric forms or mixtures and individual geometric or optical isomers, particularly racemic or non-racemic mixtures of the isomers thereof.

It is also preferable to use compounds of general formula 1 to prepare a medicament for the treatment of the above-mentioned respiratory complaints, wherein
R¹ and R² denote H or a group selected from among —C₁₋₆-alkyl, —C₃₋₈-cycloalkyl and —C₁₋₆-alkyl-O—C₁₋₆-alkyl, which may optionally be mono- or polysubstituted by one or more groups selected from among —OH, —NR⁴R⁵, morpholinyl and pyridyl or
R¹ and R² together with the nitrogen form a ring selected from among morpholinyl, thiomorpholinonyl, piperidyl and piperazinyl which may optionally be mono- or polysubstituted by one or more groups selected from among —OH, —C₁₋₆-alkyl-OH, —O—C₁₋₆-alkyl, —COOR⁴, —NR⁴R⁵, —CO—NR⁴R⁵, —COR⁶ and —NH—COR⁶;
R⁴ and R⁵ independently of one another denote H, —C₁₋₆-alkyl;
R⁶ denotes H, —C₁₋₆-alkyl, —NR⁴R⁵, —C₁₋₆-alkyl-CO—C₁₋₆-alkyl, furanyl, thiophenyl;
R³ denotes —C₁₋₈-alkyl, —C₃₋₈-cycloalkyl, —C₁₋₆-alkyl-R⁷ or phenyl, which may optionally be anellated or may be mono- or polysubstituted by one or more groups selected from among —CF₃, —COOR⁴, halogen, —NO₂, —NR⁴R⁵, —OR⁴ and —SR⁴;
R⁷ denotes furanyl, 2,3-dihydro-1H-inden-2-yl, naphthyl, 1,3-benzodioxole or phenyl, which may optionally be mono- or polysubstituted by one or more substituents selected from among —C₁₋₆-alkyl, —O—C₁₋₆-alkyl, halogen, —NO₂ and —CF₃;
X is —S—, —S(O)—, —S(O₂)—;
Y is —NR⁴—, —S—, —O—;
and the pharmacologically acceptable acid addition salts, tautomeric and isomeric forms or mixtures and individual geometric or optical isomers, particularly racemic or non-racemic mixtures of the isomers thereof.

Most preferably, compounds of general formula 1 are used to prepare a medicament for the treatment of the above-mentioned respiratory complaints, wherein
R¹ denotes C₁₋₆-alkyl, —C₃₋₈-cycloalkyl or —C₁₋₆-alkyl-O—C₁₋₆-alkyl, which may optionally be mono- or polysubstituted by one or more groups selected from among —OH, —NR⁴R⁵, morpholinyl and pyridyl or
R² denotes H or —C₁₋₆-alkyl or
R¹ and R² together with the nitrogen denote a piperazinyl ring;
R⁴ and R⁵ independently of one another denote H, —C₁₋₆alkyl;
R³ denotes —C₁₋₈-alkyl, —C₃₋₈-cycloalkyl, —C₁₋₆-alkyl-R⁷ or phenyl, which may optionally be anellated or may be mono- or polysubstituted by one or more groups selected from among —CF₃, —COOR⁴, halogen, —NO₂, —NR⁴R⁵, —OR⁴ and —SR⁴;
R⁷ denotes furanyl, 2,3-dihydro-1H-inden-2-yl, naphthyl, 1,3-benzodioxole or phenyl, which may optionally be mono- or polysubstituted by one or more substituents selected from among —C₁₋₆-alkyl, —O—C₁₋₆-alkyl, halogen, —NO₂ and —CF₃;
X is —S—, —S(O)—, —S(O₂)—;
Y is —S—;
and the pharmacologically acceptable acid addition salts, tautomeric and isomeric forms or mixtures and individual geometric or optical isomers, particularly racemic or non-racemic mixtures of the isomers thereof.

Of these, the compounds numbered 2-17 are particularly preferred, the asterisk * indicating the point of connection to the pyrimidopyrimidine A.

| | |
|---|---|
| | A |
| 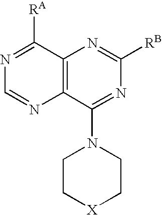 | |

| Num- | | | |
|---|---|---|---|
| ber | R^{A} | R^{B} | X |
| 2 | 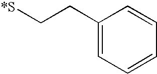 | 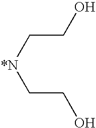 | S═O |
| 3 | 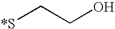 | 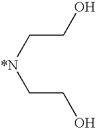 | S═O |
| 4 | 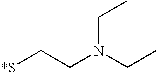 | 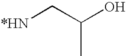 | S═O |
| 5 | 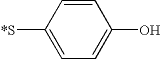 | 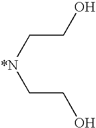 | S═O |
| 6 | 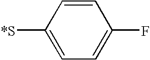 | 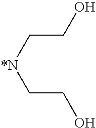 | S═O |
| 7 | 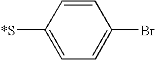 | 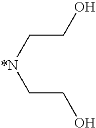 | S═O |
| 8 | 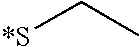 | 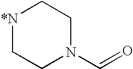 | S═O |
| 9 | 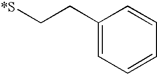 | 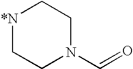 | S═O |
| 10 | 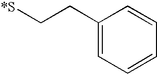 | 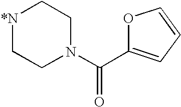 | S═O |
| 11 | 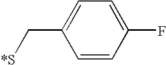 | 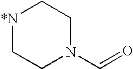 | S═O |
| 12 | 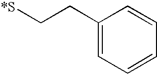 | 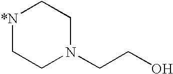 | S═O |
| 13 | *SMe | 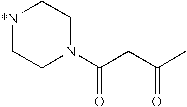 | S |
| 14 | *SMe | 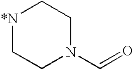 | S |
| 15 | *SMe | 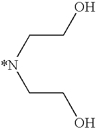 | S═O |
| 16 | *SMe | 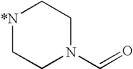 | S═O |
| 17 | *SMe | 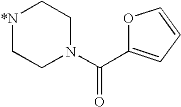 | S═O |

It is preferable to use compounds of general formula 1 to prepare a medicament for the treatment of the above-mentioned respiratory complaints, wherein
R¹ denotes C₁₋₆-alkyl, —C₃₋₈-cycloalkyl or —C₁₋₆-alkyl-O—C₁₋₆-alkyl, which may optionally be mono- or polysubstituted by one or more groups selected from among —OH, —NR⁴R⁵, morpholinyl and pyridyl;
R² denotes H or —C₁₋₆-alkyl or
R¹ and R² together with the nitrogen denote a piperazinyl ring;
R⁴ and R⁵ independently of one another denote H, —C₁₋₆-alkyl;
R³ denotes benzyl;
X is —S—, —S(O)—, —S(O₂)—; Y is —S—;
and the pharmacologically acceptable acid addition salts, tautomeric and isomeric forms or mixtures and individual geometric or optical isomers, particularly racemic or non-racemic mixtures of the isomers thereof.

Of these, compounds numbered 18-65 are particularly preferred, the asterisk * indicating the point of connection to the pyrimidopyrimidine A.

| Num- | | | |
|---|---|---|---|
| ber | R^{A} | R^{B} | X |
| 18 | 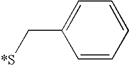 | 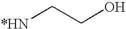 | 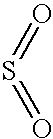 |
| 19 | 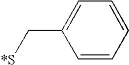 | 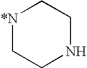 | 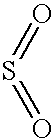 |
| 20 | 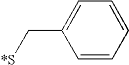 | 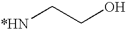 | S |
| 21 | 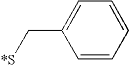 | 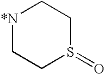 | S |
| 22 | 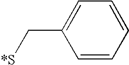 | 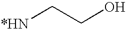 | S═O |
| 23 | 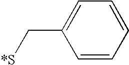 | 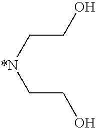 | S═O |
| 24 | 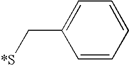 | 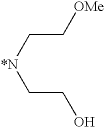 | S═O |
| 25 | 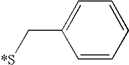 | 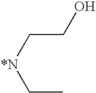 | S═O |
| 26 | 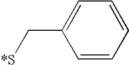 | 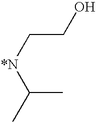 | S═O |
| 27 | 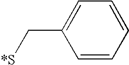 | 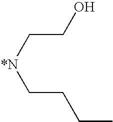 | S═O |
| 28 | 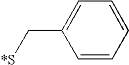 | 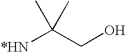 | S═O |
| 29 | 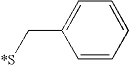 | 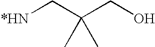 | S═O |
| 30 | 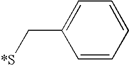 | 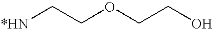 | S═O |
| 31 | 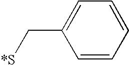 | 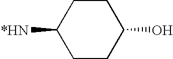 | S═O |
| 32 | 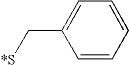 | 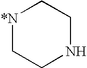 | S |
| 33 | 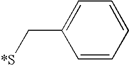 | 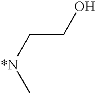 | S═O |
| 34 | 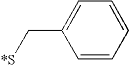 | 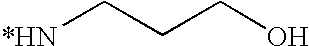 | S═O |
| 35 | 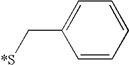 | 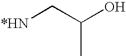 | S═O |
| 36 | 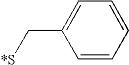 | 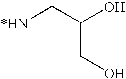 | S═O |
| 37 | 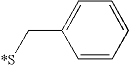 | 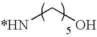 | S═O |
| 38 | 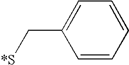 | 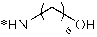 | S═O |
| 39 | 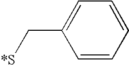 | 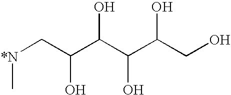 | S═O |
| 40 | 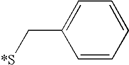 | 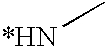 | S═O |
| 41 | 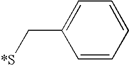 | 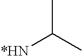 | S═O |
| 42 | 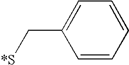 | 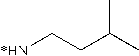 | S═O |
| 43 | 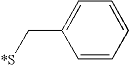 | 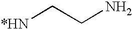 | S═O |
| 44 | 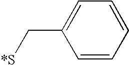 | 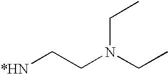 | S═O |
| 45 | 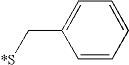 | 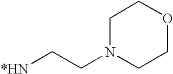 | S═O |
| 46 | 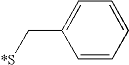 | 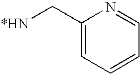 | S═O |
| 47 | 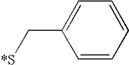 | 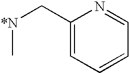 | S═O |
| 48 | 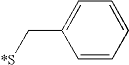 | 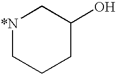 | S═O |
| 49 | 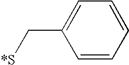 | 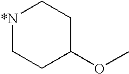 | S═O |
| 50 | 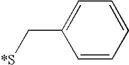 | 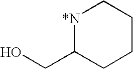 | S═O |
| 51 | 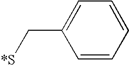 | 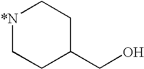 | S═O |
| 52 | 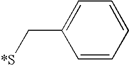 | 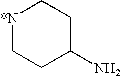 | S═O |
| 53 | 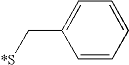 | 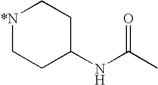 | S═O |
| 54 | 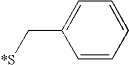 | 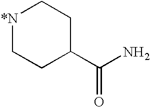 | S═O |
| 55 | 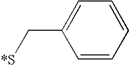 | 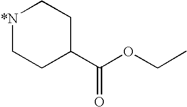 | S═O |
| 56 | 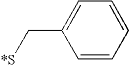 | 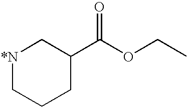 | S═O |
| 57 | 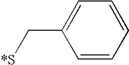 | 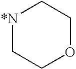 | S═O |
| 58 | 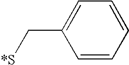 | 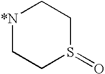 | S═O |
| 59 | 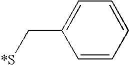 | 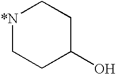 | S═O |
| 60 | 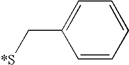 | 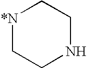 | S═O |
| 61 | 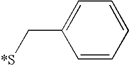 | 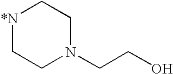 | S═O |
| 62 | 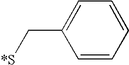 | 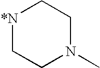 | S═O |
| 63 | 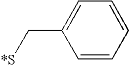 | 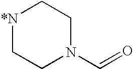 | S═O |
| 64 | 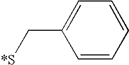 | 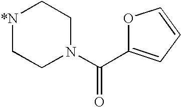 | S═O |
| 65 | 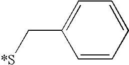 | 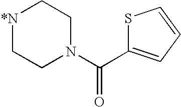 | S═O |

It is also preferable to use compounds of general formula 1 to prepare a medicament for the treatment of the above-mentioned respiratory complaints, wherein
R¹ and R² together with the nitrogen denote a piperazinyl ring;
R³ denotes —C₁₋₈-alkyl, —C₃₋₈-cycloalkyl, —C₁₋₆-alkyl-R⁷ or phenyl, which may optionally be anellated or may be mono- or polysubstituted by one or more groups selected from among —CF₃, —COOR⁴, halogen, —NO₂, —NR⁴R⁵, —OR⁴ and —SR⁴;
R⁷ denotes furanyl, 2,3-dihydro-1H-inden-2-yl, naphthyl, 1,3-benzodioxole or phenyl, which may optionally be mono- or polysubstituted by one or more substituents selected from among —C₁₋₆-alkyl, —O—C₁₋₆-alkyl, halogen, —NO₂ and —CF₃;
X is —S—, —S(O)—, —S(O₂)—;
Y is —S—;
as well as pharmacologically acceptable acid addition salts, tautomeric and isomeric forms or mixtures and individual geometric or optical isomers, particularly racemic or non-racemic mixtures of the isomers thereof.

Of these, compounds numbered 66-95 are particularly preferred, the asterisk * indicating the point of connection to the pyrimidopyrimidine A.

| Number | R^{A} | R^{B} | X |
|---|---|---|---|
| 66 | 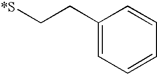 | 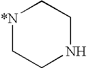 | S |
| 67 | 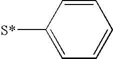 | 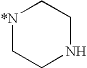 | S |
| 68 | 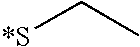 | 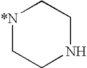 | S═O |
| 69 | 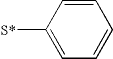 | 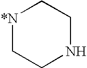 | S═O |
| 70 | 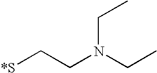 | 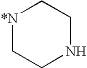 | S═O |
| 71 | 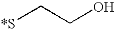 | 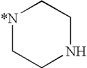 | S═O |
| 72 | 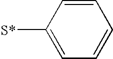 | 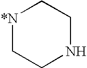 | S═O |
| 73 | 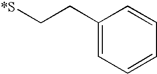 | 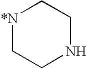 | S═O |
| 74 | 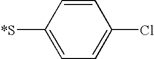 | 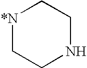 | S═O |
| 75 | 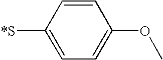 | 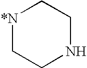 | S═O |
| 76 | 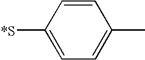 | 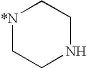 | S═O |
| 77 | 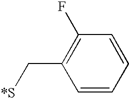 | 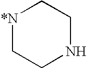 | S═O |
| 78 | 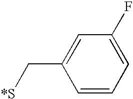 | 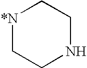 | S═O |
| 79 | 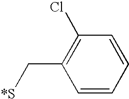 | 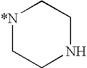 | S═O |
| 80 | 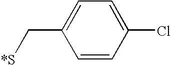 | 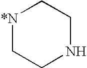 | S═O |
| 81 | 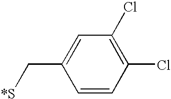 | 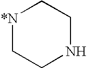 | S═O |
| 82 | 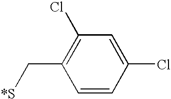 | 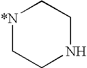 | S═O |
| 83 | 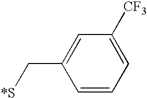 | 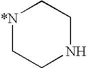 | S═O |
| 84 | 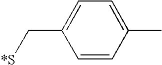 | 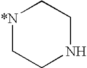 | S═O |
| 85 | 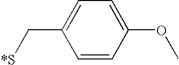 | 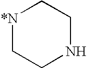 | S═O |
| 86 | 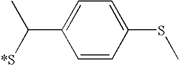 | 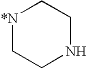 | S═O |
| 87 | 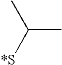 | 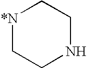 | S═O |
| 88 | 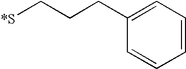 | 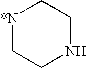 | S═O |
| 89 | 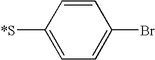 | 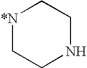 | S═O |
| 90 | 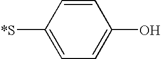 | 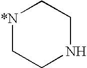 | S═O |
| 91 | 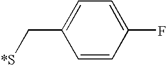 | 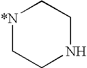 | S═O |
| 92 | 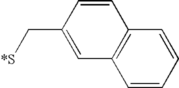 | 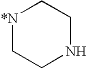 | S═O |
| 93 | 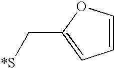 | 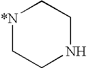 | S═O |
| 94 | *SMe | 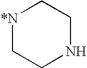 | S |
| 95 | *SMe | 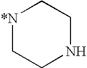 | S═O |

It is also preferable to use compounds of general formula 1 to prepare a medicament for the treatment of the above-mentioned respiratory complaints, wherein
R¹ denotes —CH₂—CH₂—OH;
R² denotes H;
R³ denotes —C₁₋₈-alkyl, —C₃₋₈-cycloalkyl, —C₁₋₆-alkyl-R⁷ or phenyl, which may optionally be anellated or may be mono- or polysubstituted by one or more groups selected from among —CF₃, —COOR⁴, halogen, —NO₂, —NR⁴R⁵, —OR⁴ and —SR⁴;
R⁷ denotes furanyl, 2,3-dihydro-1H-inden-2-yl, naphthyl, 1,3-benzodioxole or phenyl, which may optionally be mono- or polysubstituted by one or more substituents selected from among —C₁₋₆-alkyl, —O—C₁₋₆-alkyl, halogen, —NO₂ and —CF₃;
X is —S—, —S(O)—, —S(O₂)—;
Y is —S—;
as well as pharmacologically acceptable acid addition salts, tautomeric and isomeric forms or mixtures and individual geometric or optical isomers, particularly racemic or non-racemic mixtures of the isomers thereof.

Of these, the compounds numbered 96-127 are particularly preferred, the asterisk * indicating the point of connection to the pyrimidopyrimidine A.

| Number | R^{A} | R^{B} | X |
|---|---|---|---|
| 96 | 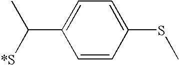 | 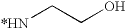 | S═O |
| 97 | 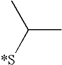 | 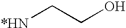 | S═O |
| 98 | 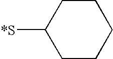 | 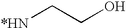 | S═O |
| 99 | 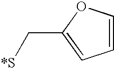 | 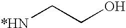 | S═O |
| 100 | 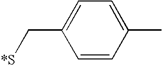 | 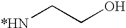 | S═O |
| 101 | 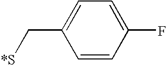 | 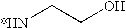 | S═O |
| 102 | 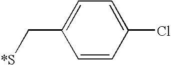 | 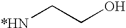 | S═O |
| 103 | 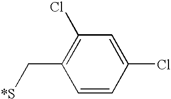 | 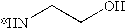 | S═O |
| 104 | 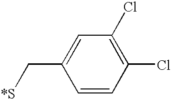 | 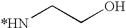 | S═O |
| 105 | 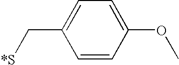 | 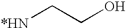 | S═O |
| 106 | 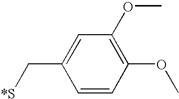 | 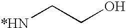 | S═O |
| 107 | 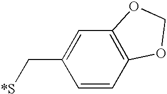 | 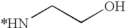 | S═O |
| 108 | 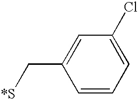 | 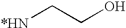 | S═O |
| 109 | 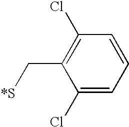 | 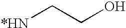 | S═O |
| 110 | 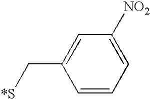 | 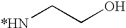 | S═O |
| 111 | 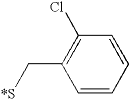 | 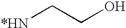 | S═O |
| 112 | 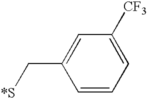 | 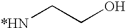 | S═O |
| 113 | 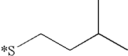 | 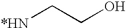 | S═O |
| 114 | 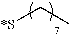 | 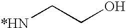 | S═O |
| 115 | 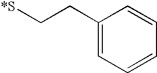 | 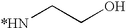 | S═O |
| 116 | 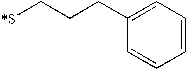 | 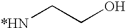 | S═O |
| 117 | 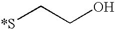 | 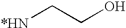 | S═O |
| 118 | 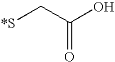 | 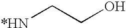 | S═O |
| 119 | 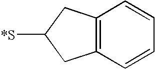 | 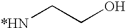 | S═O |
| 120 | 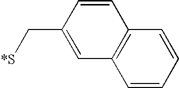 | 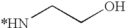 | S═O |
| 121 | 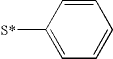 | 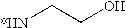 | S═O |
| 122 | 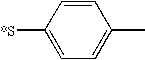 | 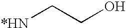 | S═O |
| 123 | 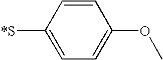 | 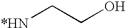 | S═O |
| 124 | 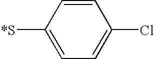 | 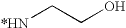 | S═O |
| 125 | 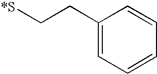 | 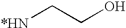 | S═O |
| 126 | 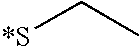 | 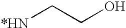 | S═O |
| 127 | *SMe | 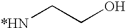 | S═O |

In another aspect the invention relates to medicaments for the treatment of respiratory complaints which contain one or more of the above-mentioned pyrimido[5,4-d]pyrimidines of general formula 1, which are used in combination with one or more additional active substances selected from among the anticholinergics, steroids or β-agonists, together or successively, for simultaneous, sequential or separate administration.

Therefore, pharmaceutical formulations are preferred which are characterised in that they contain one or more compounds of formula 1 according to the preferred embodiments described above.

Preferably the present invention relates to the use of compounds of general formula 1 for preparing a pharmaceutical composition for the treatment of inflammatory or obstructive diseases of the upper and lower respiratory organs including the lungs, such as for example allergic rhinitis, chronic rhinitis, bronchiectasis, cystic fibrosis, asthma, COPD, idiopathic pulmonary fibrosis and fibrosing alveolitis.

The compounds of general formula 1 may be used on their own or in combination with other compounds of general formula 1 according to the invention, optionally also in combination with other pharmacologically active substances. Examples of other pharmacologically active substances might be e.g. anticholinergics (ipratropium, oxitropium, tiotropium), steroids or β₂-agonists (albuterol, salmeterol, formoterol).

Suitable preparations include for example tablets, capsules, solutions, syrups, emulsions or inhalable powders or aerosols. The content of the pharmaceutically active compound(s) in each case should be in the range from 0.1 to 90 wt.-%, preferably 0.5 to 50 wt.-% of the composition as a whole, i.e. in amounts which are sufficient to achieve the dosage range specified below.

Oral administration may be in the form of a tablet, in the form of a powder, powder in a capsule (e.g. a hard gelatine capsule), a solution or suspension. If the substance is administered by inhalation the active substance combination may be taken as a powder, as an aqueous or aqueous-ethanolic solution or by means of a propellant gas formulation.

Preferably, the compounds of general formula 1 are administered orally, and it is particularly preferable if they are administered once or twice a day. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatine capsules. Suitable suppositories may be made for example by mixing with carriers provided for this purpose, such as neutral fats or polyethyleneglycol or the derivatives thereof. Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

It is also preferable if the compounds of general formula 1 are administered by inhalation and it is particularly preferable if they are administered once or twice a day. For this the compounds of general formula 1 have to be prepared in inhalable formulations. Suitable inhalable formulations include inhalable powders, propellant gas-containing metered dose aerosols or propellant-free inhalable solutions, which are optionally admixed with conventional physiologically acceptable excipients.

Within the scope of the present invention the term propellant-free inhalable solutions also includes concentrates or sterile, ready-to-use inhalable solutions. The preparations which may be used within the scope of the present invention are described in detail in the next section of the description.

Inhalable Powders

If the compounds of general formula 1 are present in admixture with physiologically acceptable excipients, the following physiologically acceptable excipients may be used to prepare the inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred. Processes for preparing the inhalable powders according to the invention by grinding and micronising and lastly mixing the ingredients together are known from the prior art.

Propellant-Containing Inhalable Aerosols

The propellant-containing inhalable aerosols which may be used within the scope of the invention may contain 1 dissolved in the propellant gas or in dispersed form. The propellant gases used to prepare the inhalable aerosols are known from the prior art. Suitable propellant gases are selected from among hydrocarbons such as n-propane, n-butane or isobutane and halohydrocarbons such as preferably fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG134a (1,1,1,2-tetrafluoroethane), TG227 (1,1,1,2,3,3,3-heptafluoropropane) and mixtures thereof. The propellant-driven inhalation aerosols which may be used according to the invention may also contain other ingredients such as co-solvents, stabilisers, surfactants, antioxidants, lubricants and pH adjusters. All these ingredients are known in the art.

Propellant-Free Inhalable Solutions

The use of compounds of general formula 1 according to the invention is preferably with the intention of preparing propellant-free inhalable solutions and suspensions. Suitable solvents for this purpose include aqueous or alcoholic, preferably ethanolic solutions. The solvent may consist exclusively of water or may be a mixture of water and ethanol. The solutions or suspensions are adjusted to a pH of 2 to 7, preferably 2 to 5, with suitable acids. The pH may be adjusted using acids selected from inorganic or organic acids. Examples of particularly suitable inorganic acids are hydrochloric acid, hydrobromic acid, nitric acid, sulphuric acid and/or phosphoric acid. Examples of particularly suitable organic acids are: ascorbic acid, citric acid, malic acid, tartaric acid, maleic acid, succinic acid, fumaric acid, acetic acid, formic acid and/or propionic acid etc. Preferred inorganic acids are hydrochloric acid, sulphuric acid. It is also possible to use acids which form an acid addition salt with one of the active substances. Of the organic acids, ascorbic acid, fumaric acid and citric acid are preferred. If desired, mixtures of the above-mentioned acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

Co-solvents and/or other excipients may be added to the propellant-free inhalable solutions which may be used according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body. Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art.

In another aspect the invention relates to a method of treating respiratory complaints by means of pyrimido[5,4-d]pyrimidines, particularly while reducing side-effects such as emesis or nausea.

For this it provides a ready-to-use package of a medicament for the treatment of respiratory complaints, containing an enclosed description which contains words selected from among respiratory complaint, COPD or asthma, a pyrimido[5,4-d]pyrimidine and one or more combination partners selected from among the anticholinergics, steroids or β-agonists.

Terms and Definitions Used

By pharmacologically acceptable acid addition salts are meant, for example, the salts selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Unless otherwise stated, C₁₋₆-alkyl groups are straight-chain or branched alkyl groups having 1 to 6 carbon atoms. The following are mentioned by way of example: methyl, ethyl, propyl or butyl. In some cases the abbreviations Me, Et, Prop or Bu are used to denote the groups methyl, ethyl, propyl or butyl. Unless otherwise stated, the definitions propyl and butyl include all the possible isomeric forms of the groups in question. Thus, for example, propyl includes n-propyl and iso-propyl, butyl includes iso-butyl, sec.butyl and tert.-butyl, etc. Methyl, ethyl, n-propyl, iso-propyl, iso-butyl, sec-butyl and tert-butyl are preferred.

The term C₃₋₈-cycloalkyl with 3-8 carbon atoms denotes for example cyclopropyl, cyclobutyl, cyclopentyl, cyclohexyl, cycloheptyl or cyclooctyl. Cyclopropyl and cyclohexyl are preferred.

Within the scope of the present invention halogen denotes fluorine, chlorine, bromine or iodine. Unless stated otherwise, fluorine, chlorine and bromine are the preferred halogens.

The term aryl denotes an aromatic ring system with 6 to 10 carbon atoms. Preferred aryl groups are phenyl or naphthyl, while the cyclic group may be substituted as specified in the definitions.

By heteroaryl rings (also abbreviated to heteroaryl) are meant, within the scope of the present invention, aromatic ring systems which contain one, two or three heteroatoms selected from among oxygen, nitrogen and sulphur and are optionally substituted as hereinbefore defined. The following are mentioned by way of example: furan, thiophene, pyrrole, pyrazole, imidazole, pyridine, pyrimidine, triazine, oxazole, isoxazole, thiazole, isothiazole, oxadiazole and pyrazolidine, while the heterocycle may be substituted as specified in the definitions.

Examples of 5- or 6-membered saturated or unsaturated heterocyclic rings (also abbreviated to heterocycle), which may contain as heteroatoms one, two or three heteroatoms selected from among oxygen, nitrogen and sulphur, include for example furan, tetrahydrofuran, 2-methyltetrahydrofuran, 2-hydroxymethylfuran, tetrahydrofuranone, γ-butyrolactone, α-pyran, γ-pyran, dioxolane, tetrahydropyran, dioxane, thiophene, dihydrothiophene, thiolane, dithiolane, pyrrole, pyrroline, pyrrolidine, pyrazole, pyrazoline, imidazole, imidazoline, imidazolidine, triazole, tetrazole, pyridine, piperidine, pyridazine, pyrimidine, pyrazine, piperazine, triazine, tetrazine, morpholine, thiomorpholine, oxazole, isoxazole, oxazine, thiazole, isothiazole, thiadiazole, oxadiazole, pyrazolidine, while the heterocycle may be substituted as specified in the definitions.

Examples of 5-, 6- or 7-membered, saturated or unsaturated, heterocyclic rings which may be formed by the groups R¹ and R² together with the nitrogen include: pyrrole, pyrroline, pyrrolidine, 2-methylpyrrolidine, 3-methylpyrrolidine, piperidine, piperazine, N-methylpiperazine, N-ethylpiperazine, N-(n-propyl)-piperazine, N-benzylpiperazine, morpholine, thiomorpholine, imidazole, imidazoline, imidazolidine, pyrazole, pyrazoline, pyrazolidine, preferably morpholine, N-benzylpiperazine, piperazine, and piperidine, while the heterocycles mentioned may be substituted as specified in the definitions. Particularly preferred rings in this context are: pyrrole, piperidine, piperazine, N-methylpiperazine, N-benzylpiperazine, morpholine, imidazole, imidazoline, imidazolidine, pyrazole, pyrazoline, pyrazolidine, preferably morpholine, N-benzylpiperazine, piperazine, and piperidine, while the heterocycles mentioned may be substituted as specified in the definitions.

By respiratory complaints are meant, within the scope of the invention, disorders which cause a patient breathing difficulties, respiratory distress or pain in the airways, particularly inflammatory or obstructive respiratory complaints. Reference is preferably made to inflammatory or obstructive diseases of the upper and lower respiratory organs including the lungs, such as for example allergic rhinitis, chronic rhinitis, bronchiectasis, cystic fibrosis, asthma, COPD, idiopathic pulmonary fibrosis and fibrosing alveolitis. Reference is made particularly to asthma, chronic bronchitis or COPD.

By reduced side-effects is meant, within the scope of the invention, the ability to administer a dose of a pharmaceutical composition without causing the patient to suffer vomiting or, better still, nausea, particularly preferably without causing any malaise. Most preferably a therapeutically effective amount of a substance can be administered without triggering emesis or nausea at any stage of the course of the disease.

1. A method of treating respiratory complaints comprised of the step of administering to a patient in need thereof a therapeutically effective amount of a medicament comprised of a substituted pyrimido[5,4-d]pyrimidine or a physiologically acceptable salt thereof.

2. The method of claim 1 wherein the respiratory complaint is an inflammatory or obstructive respiratory complaint.

3. The method of claim 2 wherein the respiratory complaint is COPD or Asthma.

4. The method of claim 1 wherein the side-effects of said treatment are reduced.

5. The method of claim 4 wherein the reduced side effects are chosen from emesis and nausea.

6. The method of claim 1 wherein the medicament is administered once or twice a day.

7. The method of claim 1, wherein the pyrimido[5,4-d]pyrimidine is a compound of general formula 1, 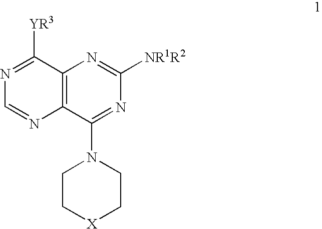
wherein
R¹ and R² independently of one another denote H, —C₁₋₆alkyl, —C₃₋₈-cycloalkyl, —C₁₋₆alkyl-O—C₁₋₆-alkyl, in each case optionally substituted by one or more substituents selected from the group aryl, —CF₃, —CN, —CONR⁴R⁵, —COOR⁴, —COR⁶, halogen, heteroaryl, heterocycle, —NO₂, —NR⁴COR⁶, —NR⁴R⁵, —NR⁴SO₂R⁴, —OR⁴, —SO₂NR⁴R⁵, —SO₂R⁴, —SOR⁴ or —SR⁴; or
R¹ and R² together with the nitrogen form a ring which may optionally be substituted by one or more substituents selected from among aryl, —C₁₋₆-alkyl-OH, —CF₃, —CN, —CONR⁴R⁵, —COOR⁴, —COR⁶, halogen, heteroaryl, heterocycle, —NO₂, —NR⁴COR⁶, —NR⁴R⁵, —NR⁴SO₂R⁴, —O—C₁₋₆-alkyl, —OR⁴, —SO₂NR⁴R⁵, —SO₂R⁴, —SOR⁴ or —SR⁴;
R⁴ and R⁵ independently of one another are H, —C₁₋₆-alkyl;
R⁶ is H, —C₁₋₆-alkyl, —C₁₋₆-alkyl-CO—C₁₋₆-alkyl, heteroaryl, heterocycle, —NR⁴R⁵;
R³ is H, —C₁₋₈-alkyl, —C₃₋₈-cycloalkyl, —C₁₋₆-alkyl-phenyl or phenyl, wherein each group may optionally be substituted or if possible anellated with one or more substituents selected from among —C₁₋₆-alkyl-OH, —CF₃, —CN, —CONR⁴R⁵, —COOR⁴, —COR⁴, halogen, heteroaryl, heterocycle, —NO₂, —NR⁴COR⁴, —NR⁴R⁵, —NR⁴SO₂R⁴, —O—C₁₋₆-alkyl, —OR⁴, —SO₂NR⁴R⁵, —SO₂R⁴, —SOR⁴ or —SR⁴;
X is —S—, —S(O)—, —S(O₂)—;
Y is —NR⁴—, —S—, —O—;
and the pharmacologically acceptable acid addition salts, tautomeric and isomeric forms or mixtures and individual geometric or optical isomers, particularly racemic or non-racemic mixtures of the isomers thereof.

8. The method according to claim 7 wherein the substituted pyrimido[5,4-d]pyrimidine is a compound of general formula 1 and;
R¹ and R² independently of one another are H, —C₁₋₆-alkyl, —C₃₋₈-cycloalkyl, —C₁₋₆-alkyl-O—C₁₋₆-alkyl, in each case optionally substituted by one or more substituents selected from among —CONR⁴R⁵, —COOR⁴, —COR⁶, halogen, heteroaryl, heterocycle, —NR⁴COR⁶, —NR⁴R⁵, —OR⁴; or
R¹ and R² together with the nitrogen form a ring which may optionally be substituted by one or more substituents selected from among —C₁₋₆-alkyl-OH, —CONR⁴R⁵, —COOR⁴, —COR⁶, —NR⁴COR⁶, —NR⁴R⁵, —NR⁴SO₂R⁴, —O—C₁₋₆-alkyl, —OR⁴, —SO₂NR⁴R⁵, —SO₂R⁴, —SOR⁴ or —SR⁴; or
R⁴ and R⁵ independently of one another are H, —C₁₋₆-alkyl;
R⁶ is H, —C₁₋₆-alkyl, —C₁₋₆-alkyl-CO—C₁₋₆-alkyl, heteroaryl, heterocycle, —NR⁴R⁵;
R³ is H, —C₁₋₈-alkyl, —C₃₋₈-cycloalkyl, —C₁₋₆-alkyl-R⁷ or phenyl, optionally substituted by one or more substituents selected from among —C₁₋₆-alkyl-OH, —CF₃, —CN, —CONR⁴R⁵, —COOR⁴, —COR⁴, halogen, —NO₂, —NR⁴COR⁴, —NR⁴R⁵, —NR⁴SO₂R⁴, —O—C₁₋₆-alkyl or —OR⁴;
R⁷ is —OH, COOR⁴, —NR⁴R⁵, heteroaryl, heterocycle, heteroaryl anellated with heterocycle, phenyl optionally substituted by one or more substituents selected from among —C₁₋₆-alkyl, —O—C₁₋₆-alkyl, halogen, —NO₂, —CF₃;
X is —S—, —S(O)—, —S(O₂)—;
Y is —NR⁴—, —S—, —O—;
and the pharmacologically acceptable acid addition salts, tautomeric and isomeric forms or mixtures and individual geometric or optical isomers, particularly racemic or non-racemic mixtures of the isomers thereof.

9. The method according to claim 7 wherein the substituted pyrimido[5,4-d]pyrimidine is a compound of general formula 1 and
R¹ and R² independently of one another are H, —C₁₋₆-alkyl, —C₃₋₈-cycloalkyl, —C₁₋₆-alkyl-O—C₁₋₆-alkyl, in each case optionally substituted by one or more substituents selected from among —OH, —NR⁴R⁵, morpholinyl, pyridyl; or
R¹ and R² together with the nitrogen form a ring selected from among morpholinyl, thiomorpholinonyl, piperidyl, piperazinyl, in each case optionally substituted by one or more substituents selected from among —OH, —C₁₋₆-alkyl-OH, —O—C₁₋₆-alkyl, —COOR⁴, —NR⁴R⁵, —CO—NR⁴R⁵, —COR⁶, —NH—COR⁶;
R⁴ and R⁵ independently of one another are H, —C₁₋₆-alkyl;
R⁶ is H, —C₁₋₆-alkyl, —NR⁴R⁵, —C₁₋₆-alkyl-CO—C₁₋₆-alkyl, furanyl, thiophenyl;
R³ is —C₁₋₈-alkyl, —C₃₋₈-cycloalkyl, —C₁₋₆-alkyl-R⁷, phenyl optionally substituted by one or more substituents selected from among —C₁₋₆alkyl, —O—C₁₋₆-alkyl, —S—C₁₋₆-alkyl, halogen, —NO₂, —CF₃;
R⁷ is —OH, COOR⁴, —NR⁴R⁵, furanyl, 2,3-dihydro-1H-inden-2-yl, naphthyl, 1,3-benzodioxole, phenyl optionally substituted by one or more substituents selected from among —C₁₋₆-alkyl, —O—C₁₋₆-alkyl, halogen, —NO₂, —CF₃;
X is —S—, —S(O)—, —S(O₂)—;
Y is —NR⁴—, —S—, —O—;
and the pharmacologically acceptable acid addition salts, tautomeric and isomeric forms or mixtures and individual geometric or optical isomers, particularly racemic or non-racemic mixtures of the isomers thereof.

10. A method of treating respiratory complaints comprised of the step of administering to a patient in need thereof a thereapuetically effective amount of medicament containing 1 to 200 mg of an active substance of general formula 1, according to one general formula 1 of claim 7 or pharmacologically acceptable acid addition salts thereof.

11. A method of treating respiratory complaints to a patient in need thereof comprised of the step of successive, simultaneous, sequential or separate administration of a medicament comprised of one or more compounds of formula 1 according to claim 7, in combination with one or more additional active substances selected from one or more among the anticholinergics, steroids or β-agonists.
